(19) United States
(12) Patent Application Publication
(10) Pub. No.: US 2026/0273247 A1
(43) Pub. Date: Sep. 17, 2026

(54) LUNG AIRWAY BYPASS STENT AND METHOD FOR IMPLANTING AND USING THE STENT TO TREAT EMPHYSEMA

(71) Applicant: DEERFIELD CATALYST, LLC, New York, NY (US)

(72) Inventors: Shashank RAINA, New York, NY (US); Don TANAKA, New York, NY (US); Zoar ENGELMAN, New York, NY (US); Mark GELFAND, New York, NY (US)

(21) Appl. No.: 19/469,219

(22) PCT Filed: Mar. 29, 2024
(86) PCT No.: PCT/US2024/022150

§ 371 Date: Sep. 25, 2025

Related U.S. Application Data
(60) Provisional application No. 63/455,496, filed on Mar. 29, 2023.

(51) Int. Cl. A61M 27/00

(52) U.S. Cl. CPC A61M 27/008; A61M 2210/1035

(57) ABSTRACT

A method for implanting a stent in an airway of a lung includes forming an opening in a wall of an airway of the lung and advancing a delivery tool through the airway wherein a distal end region of the delivery tool includes a catheter on which the stent, in a compressed configuration, is mounted and a sleeve extending over the catheter and stent. Also the distal end region of the delivery tool is advanced from the airway and at least partially through the opening in the wall of the airway and into lung tissue adjacent the wall. The stent is positioned in the opening by manipulating the distal end of the delivery tool. The sleeve is moved or the catheter is advanced to move a first flange of the stent to extend from the sleeve and be positioned in the lung tissue and to allow the first flange to at least partially expand from the compressed configuration. The sleeve is further moved or the catheter is further advanced such that stent is beyond the sleeve and the stent further expands from the compressed configuration into an expanded configuration.

### CROSS-REFERENCES TO RELATED APPLICATIONS

This application claims the benefit of U.S. Provisional Applications 63/455,496, filed Mar. 29, 2023, which is incorporated herein by reference in its entirety.

### FIELD OF INVENTION

The invention is in the field of treating emphysema and other diseases of the lung by artificially creating air passages in a lung to vent or drain fluids from a diseased portion of a lung.

### BACKGROUND

Lung diseases include Chronic Obstructive Pulmonary Disorder (COPD), emphysema, chronic bronchitis, and asthma. These diseases damage lungs such that diseased lung portions are unable to effectively exchange oxygen and carbon dioxide between blood and air. Because of the diseased lung portions, other portions of the lung, which remain functional, do not receive as much air as they are capable of handling.

The lungs are part of the lower respiratory airway tract (passage) that begins at the trachea and branches into the bronchi and bronchioles which direct air to and from the alveoli. Gas exchange occurs in the alveoli. The pair of lungs in a typical human contain approximately 2,400 kilometres (1,500 miles) of airways and 300 to 500 million alveoli. The lungs receive deoxygenated blood from the heart and direct the blood to the alveoli where the blood receives oxygen and expels carbon dioxide with the air in the alveoli. The parenchyma of the lung includes all tissue in the lung including bronchioles, bronchi, blood vessels, interstitium, and alveoli. Air must reach the alveoli for the gas exchange to properly occur. However, lung diseases reduce the capacity of a lung and the ability of the lung to direct the air to the alveoli. For example, lung diseases can destroy the lung parenchyma and reduce the ability of the alveoli to dilate, which can cause debilitating shortness of breath (dyspnea).

4.7 million Americans have been diagnosed with emphysema, which is chronic, progressive, irreversible, and ultimately terminal. Emphysema is characterized by an inability to breathe (dyspnea) due to the destruction of lung tissue (parenchyma). Because of the destroyed tissue, airways in the lung collapse during exhalation due to lack of support from the destroyed surrounding lung tissue. The collapse of airways leads to air being trapped in diseased areas of the lung. The diseased area remains inflated with the trapped air, which prevents functional portions of the lung from being fully inflated as air is inhaled into the lung. The trapping of air in diseased portions of a lung results in hyperinflation of the lung, which prevents the lung from receiving sufficient air during inhalation. A person with a hyperinflation suffers from not being able to inhale sufficient amounts of air and feels out of breath.

### SUMMARY

There is a long felt need for non-invasive treatments that reduce the ill effects of lung diseases and allow patients to breathe easier when suffering from these diseases. The inventors developed a treatment that uses a tool (e.g., a bronchoscope) to advance a catheter into an air passage of a lung, pierce a wall of the air passage near a diseased lung portion and implant a stent in the wall opening. The stent allows air and other fluids to vent or drain from lung tissue, through the stent into the air passage. The air and other fluids are breathed or coughed out of the air passage, through the trachea and out the mouth or nose. By venting or draining air and fluids the diseased lung portions deflate. The non-diseased portions can then more fully expand to increase the capacity of the lung to take in a breath and exchange gases. The goal of the treatment is to allow the patient to breathe easier, take in more air and no longer always feel out of breath.

The treatment may be directed towards peripheral lung areas where lung parenchyma destruction is high and perfusion is low (e.g., spider web lung). The treatment also minimizes trauma to the lung parenchyma that could cause inflammation and tissue growth that can block the bypass.

The design of the stent facilitates mechanical retention within the lung and the normal clearance of mucus by cough. The stent also increases the surface area and durability of the airway passages for gas to escape by creating one or multiple bypass pathways.

In one aspect of the technology, a stent is configured to extend through a wall of an airway of a lung to vent air from airway of the lung, the stent comprising: a hollow middle section that forms a through passage along a central longitudinal axis; a first flange located at and extending outwardly from a first end of the hollow middle section; and a second flange located at and extending outwardly from a second end of the hollow middle section that is opposite the first end, wherein the first and second flanges are asymmetrical.

The first flange may have a larger cross-section than the second flange. The first and second flanges may be flexible and may be biased to be in a fully extended position. The first flange may extend from the first end of the hollow middle section at a first angle relative to the central longitudinal axis, wherein the second flange extends from the second end of the hollow middle section at a second angle relative to the central longitudinal axis. The first angle may be greater than the second angle. The hollow middle section may comprise a biocompatible non-porous membrane, which is configured as a barrier to tissue growth through the hollow middle section. The biocompatible non-porous membrane may be formed from polytetrafluoroethylene (PTFE). The first and second flanges may be devoid of the non-porous membrane. The hollow middle section may be supported by a wire lattice. A fully deployed diameter of the first flange may be between about 11 mm to about 15 mm and a fully deployed diameter of the second flange may be between about 9 mm to about 14 mm. The fully deployed diameter of the first flange may be greater than the fully deployed diameter of the second flange. The first flange may form a first angle with the central longitudinal axis within a range of about 85 degrees to about 90 degrees. The second flange may form a second angle with the central longitudinal axis within a range of about 45 degrees to about 85 degrees.

In another aspect of the technology, a delivery system is configured to deliver the stent described above to a lung. The delivery system comprises: a catheter; and a sleeve enclosing the catheter, wherein the catheter is sized to extend through the through passage, and wherein sleeve is configured to maintain the stent in a compacted state.

Another aspect of the technology includes a stent configured to be placed in an airway of a lung, the stent comprising: a hollow middle section defining a through passage along an axis; a hollow inlet section connected to a first end of the middle section and having a cross sectional area increasing along the axis from the first end to an end of the hollow inlet section, wherein the hollow inlet section has a larger cross sectional area than a cross sectional area of the middle section; and a hollow outlet section connected to a second end of the middle section and having a cross sectional diameter increasing along the axis from the second end to an end of the hollow outlet section, wherein the length of the hollow outlet section is shorter than the lengths of each of the hollow middle section and the hollow inlet section, and wherein the hollow middle section is configured to engage an opening in an air passage of the lung.

The stent may further comprise a biocompatible non-porous membrane covering the hollow middle section and configured as a barrier to tissue growth through the hollow middle section, wherein the hollow inlet section and the hollow outlet sections are devoid of non-porous membrane which covers the hollow inlet and outlet sections. The cross section of the hollow middle section is at least one of circular, octagonal or curvilinear. The hollow inlet section may form a first angle with the axis which is greater than a second angle formed by the hollow outlet section with the axis. The hollow inlet section has an outermost diameter in a range of about 13 mm to about 18 mm, and the outlet section has an outermost diameter in a range of about 10 mm to about 17 mm. The first angle may be constant along the length of the hollow inlet section and/or the second angle may be constant along the length of the hollow outlet section. The first angle may increase along the length of the hollow inlet section from the hollow middle section towards a first end of the stent, and/or the second angle may increase along the length of the hollow outlet section from the hollow middle section towards a second end of the stent. The hollow inlet section may have a first length and the hollow outlet section may have a second length shorter than the first length. The stent may have a length a range of about 6 mm to about 13 mm. The inlet section may form an angle with respect to the axis that is in a range of about 85 to 95 degrees. The outlet section may form an angle with respect to the axis that is in a range of about 85 to about 75 degrees.

In another aspect of the technology, a method for delivering a stent to a diseased portion of a lung includes: forming an opening in a wall of an airway in or adjacent to the diseased portion of the lung; inserting a delivery tool loaded with a compressed stent, the delivery tool comprising a catheter enclosed by a slidable sleeve; directing the delivery tool to the opening in the wall of the airway; inserting the compressed stent into the opening in the wall of the airway; partially retracting the sleeve to allow a first flange of the stent to partially expand and engage lung tissue; determining whether the stent is positioned in a target location; and fully retracting the sleeve so that the first flange and a second flange of the stent fully expands.

The first flange may be pulled against the wall of the airway when the first flange is partially expanded. The first flange may anchor the stent to the lung tissue to prevent the stent from being sucked into the airway. The catheter may be fully retracted after the sleeve has been retracted and the stent is positioned in the target location. A diameter of the fully expanded first flange may be greater than a diameter of the fully expanded second flange. The first and second flanges may be asymmetric. The stent may be pulled back after the first flange is partially expanded to partially anchor the stent to the lung tissue. The stent may be partially movable while the first flange is partially expanded. The stent may be stationary and fully anchored to the lung tissue when the first flange is fully expanded.

In another aspect of the technology, a method for venting trapped air and/or draining trapped fluid in a diseased portion of a lung includes: forming an opening in a wall of a lung airway adjacent to or within a diseased portion of the lung; inserting a collapsed stent into the opening; and expanding the stent so that the stent is anchored in place within the opening, wherein the opening and the stent provide a pathway for fluid and/or air to vent or drain from the diseased portion of the lung into the airway.

The stent may be configured to automatically expand when positioned in a target location. The stent may be anchored to one side of the opening so that the stent is not sucked into the airway through the opening. The stent may comprise a middle passage sandwiched between a pair of flanges that are asymmetrical. A first flange of the pair of flanges may be positioned in the lung tissue and is pressed against a wall of the airway, and a second flange of the pair of flanges may be positioned inside the airway. The first flange may have a greater diameter than the second flange. The stent may be biased toward an expanded state. At least part of the stent may be encased in a wire mesh.

Another aspect of the technology includes a compressible and expandable stent configured to be placed relatively transversely in an airway of a lung, the stent comprising: a hollow middle section defining a through passage along an axis; a hollow inlet section connected to a first end of the middle section and having an cross sectional area increasing along a the axis from the first end to an end of the hollow inlet section, wherein the hollow inlet section has a larger cross sectional area than a cross sectional area of the middle section, and a hollow outlet section connected to a second end of the middle section and having a cross sectional diameter increasing along the axis from the second end to an end of the hollow outlet section; wherein the length of the hollow outlet section is shorter than the lengths of each of the hollow middle section and the hollow inlet section, and wherein the hollow middle section is configured to engage an opening in an air passage, such as a bronchus, of the lung.

The stent may include a biocompatible non-porous membrane, such as polytetrafluoroethylene (PTFE), covering the hollow middle section and configured as a barrier to tissue growth through the hollow middle section, wherein the hollow inlet section and the hollow outlet sections are devoid of non-porous membrane which covers the hollow inlet and outlet sections.

The hollow middle section may be circular in cross section at least one of circular, octagonal or curvilinear.

The hollow inlet section may form a first angle with the axis which equal to or differs, e.g., is greater, than a second angle formed by the hollow outlet section with the axis. The first angle may be 27 degrees, or in a range of 20 to 35 degrees, 22 to 32, or 26 to 30 degrees; and the second angle may be 45 degrees or in a range of 30 to 60 degrees, 35 to 55 degrees or 40 to 50 degrees. The first angle may be constant along the length of the hollow inlet section and/or the second angle may be constant along the length of the hollow outlet section. The first angle may increase along the length of the hollow inlet section from the hollow middle section towards a first end of the sent, and/or the second angle may increase along the length of the hollow outlet section from the hollow middle section towards a second end of the stent.

The hollow inlet section may have a first length and the hollow outlet section has a second length shorter than the first length.

The hollow inlet section may have an outermost diameter of 15 mm, 16 mm or in a range of 13 mm to 18 mm, 14 mm to 17 mm, or 15 mm to 16 mm, and the outlet section may have an outermost diameter of 12 mm, 14 mm or in a range of 10 mm to 17 mm, 11 mm to 16 mm or 12 mm to 14 mm.

Another aspect of the technology may include a device comprising: a longitudinal axis; a first end portion having a terminal first end diameter 15 mm, 16 mm or in a range of 13 mm to 18 mm, 14 mm to 17 mm, or 15 mm to 16 mm, the first end portion having a first length along the longitudinal axis, the first end portion being self-expanding or balloon expandable; a second end portion having a second end diameter 12 mm, 14 mm or in a range of 10 mm to 17 mm, 11 mm to 16 mm or 12 mm to 14 mm, and smaller than the terminal first end diameter, the second end portion being substantially frustoconical tapering towards central portion], the second end portion having a second length along the longitudinal axis, the second end portion being self-expanding; an intermediate portion between the first end portion and the second end portion, the intermediate portion being substantially cylindrical, the intermediate portion having a third length along the longitudinal axis, the intermediate portion being deformably expandable, the first end portion being frustoconical, the first end portion tapering between the terminal first end diameter and the intermediate portion; and a membrane material coupled to at least the intermediate portion, the device configured to provide fluid flow between a first airway lung passage in which the first end portion is anchored and a lung parenchyma external to the airway in which the second end portion is anchored, where device self expands from compressed diameter of 2 mm, in a range of 2.8 mm to 1.2 mm, 2.5 mm to 1.5 mm or 1.3 mm to 1.7 mm.

The membrane may be a biocompatible non-porous membrane, such as polytetrafluoroethylene (PTFE), covering the intermediate portion and configured as a barrier to tissue growth through the hollow middle section, wherein the first portion and the second portion may be devoid of non-porous membrane which covers the hollow inlet and outlet sections. The cross section of the intermediate section may be at least one of circular, octagonal or curvilinear. The first portion may form a first angle with an axis of the intermediate section which may be greater than a second angle formed by the second portion with the axis.

The first angle may be 27 degrees, or in a range of 20 to 35 degrees, 22 to 32, or 26 to 30 degrees; and the second angle is 45 degrees or in a range of 30 to 60 degrees, 35 to 55 degrees or 40 to 50 degrees. The first angle may be constant along the length of the first portion and/or the second angle may be constant along the length of the second portion. The first angle may increase along the length of the first portion from the intermediate portion towards a first end of the device, and/or the second angle may increase along the length of the second portion from the intermediate portion towards a second end of the device, opposite to the first end. The first portion may have a first length and the second portion section may have a second length shorter than the first length. The first portion may form an expansion angle with respect to an axis through the intermediate portion greater than an expansion angle of the second portion. The first portion and/or the second portion may overlap the intermediate portion. The first portion may form an angle with respect to an axis through the intermediate portion that is 90 degrees or in a range of 85 to 95 degrees. The second portion may form an angle with respect to an axis through the intermediate portion that is 80 degrees or in a range of 85 to 75 degrees.

Another aspect of the technology includes a device comprising: a longitudinal axis; a first end portion having a terminal first end diameter between 15 mm, 16 mm or in a range of 13 mm to 18 mm, 14 mm to 17 mm, or 15 mm to 16 mm, the first end portion having a first length along the longitudinal axis, the first end portion comprising a first metal wire mesh having a first mesh density; a second end portion having a second end diameter smaller than the terminal first end diameter, the second end portion being substantially cone shaped, the second end portion having a second length along the longitudinal axis, the second end portion comprising a second mesh having a second mesh density; an intermediate portion between the first end portion and the second end portion, the intermediate portion cylindrical between the first tapered end portion and the second tapered end portion, the intermediate portion having a third length along the longitudinal axis, the intermediate portion comprising a third wire mesh having a third mesh density less than the first mesh density and the second mesh density, the first end portion being frustoconical, the first end portion tapering between the terminal first end diameter and the intermediate portion.

The membrane may be a biocompatible non-porous membrane, such as polytetrafluoroethylene (PTFE), covering the intermediate portion and configured as a barrier to tissue growth through the hollow middle section, wherein the first portion and the second portion are devoid of non-porous membrane which covers the hollow inlet and outlet sections. The cross section of the intermediate section may be at least one of circular, octagonal or curvilinear. The first portion may form a first angle with an axis of the intermediate section which is greater than a second angle formed by the second portion with the axis. The first angle may be 27 degrees, or in a range of 20 to 35 degrees, 22 to 32, or 26 to 30 degrees; and the second angle may be 45 degrees or in a range of 30 to 60 degrees, 35 to 55 degrees or 40 to 50 degrees. The first angle may be constant along the length of the first portion and/or the second angle may be constant along the length of the second portion. The first angle may increase along the length of the first portion from the intermediate portion towards a first end of the device, and/or the second angle may increase along the length of the second portion from the intermediate portion towards a second end of the device, opposite to the first end. The first portion may have a first length and the second portion section may have a second length shorter than the first length. The first portion may form an expansion angle with respect to an axis through the intermediate portion greater than an expansion angle of the second portion. The first portion and/or the second portion may overlap the intermediate portion. The first portion may form an angle with respect to an axis through the intermediate portion that is 90 degrees or in a range of 85 to 95 degrees. The second portion may form an angle with respect to an axis through the intermediate portion that is 80 degrees or in a range of 85 to 75 degrees.

In another aspect of the invention, a device comprises: a longitudinal axis; a first end portion having a terminal first end diameter 15 mm, 16 mm or in a range of 13 mm to 18 mm, 14 mm to 17 mm, or 15 mm to 16 mm, the first end portion having a first length along the longitudinal axis; a second end portion having a second end diameter of 12 mm, 14 mm or in a range of 10 mm to 17 mm, 11 mm to 16 mm or 12 mm to 14 mm, and smaller than the terminal first end diameter, the second end portion having a second length along the longitudinal axis, wherein the second end portion is substantially frustoconical and the first end portion is substantially frustoconical; and an intermediate substantially cylindrical portion between the first end portion and the second end portion, the intermediate portion having a third length along the longitudinal axis.

The membrane may be a biocompatible non-porous membrane, such as polytetrafluoroethylene (PTFE), covering the intermediate portion and configured as a barrier to tissue growth through the hollow middle section. The first portion and the second portion may be devoid of non-porous membrane which covers the hollow inlet and outlet sections. The cross section of the intermediate section may be at least one of circular, octagonal or curvilinear. The first portion may form a first angle with an axis of the intermediate section which is greater than a second angle formed by the second portion with the axis. The first angle may be 27 degrees, or in a range of 20 to 35 degrees, 22 to 32, or 26 to 30 degrees; and the second angle may be 45 degrees or in a range of 30 to 60 degrees, 35 to 55 degrees or 40 to 50 degrees. The first angle may be constant along the length of the first portion and/or the second angle may be constant along the length of the second portion. The first angle may increase along the length of the first portion from the intermediate portion towards a first end of the device, and/or the second angle may increase along the length of the second portion from the intermediate portion towards a second end of the device, opposite to the first end. The first portion has a first length and the second portion section has a second length shorter than the first length. The first portion may form an expansion angle with respect to an axis through the intermediate portion greater than an expansion angle of the second portion. The first portion and/or the second portion may overlap the intermediate portion. The first portion may form an angle with respect to an axis through the intermediate portion that is 90 degrees or in a range of 85 to 95 degrees. The second portion may form an angle with respect to an axis through the intermediate portion that is 80 degrees or in a range of 85 to 75 degrees.

### BRIEF DESCRIPTION OF THE DRAWINGS

FIG. 1 is a schematic illustration of a lung with multiple bypass stents inserted into the walls of air passages in the lungs.

FIG. 2 is a schematic diagram of a stent configured to be inserted in a hole formed in an air passage of a lung.

FIG. 3 is a schematic diagram of a stent formed of wire and a membrane, wherein the stent is an expanded, deployed orientation.

FIG. 4 illustrates an exemplary delivery tool.

FIG. 5 is a schematic diagram of the stent shown in FIG. 3, wherein the stent is collapsed within a delivery tool.

FIG. 6 is a flow chart for an exemplary method of installing a stent and venting and/or draining gas and fluid from the lung.

FIG. 7 is a schematic diagram of another exemplary stent.

FIG. 8 is a schematic diagram of another exemplary stent.

### DETAILED DESCRIPTION OF EXAMPLE NON-LIMITING EMBODIMENTS

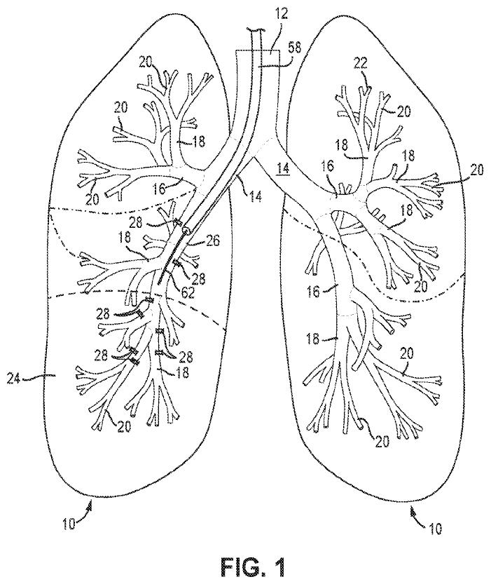

FIG. 1 shows lungs 10 connected to a trachea 12 of a human patient by way of a bronchial tree formed from the airway passages in the lungs 10. The bronchial tree includes the main bronchi 14 which connect to the trachea 12, the lobar bronchi 16, the segmental bronchi 18, the bronchioles 20, and the alveoli (not shown). The air passages become increasingly smaller and the amount of cartilage in the walls of the air passages lessens as the bronchial tree processes from the main bronchi 14, lobar bronchi 16, segmental bronchi 18, and bronchioles 20. The gas exchange between air and blood occurs in the alveoli.

In a diseased lung, diseased portions 24 of the lung can no longer exchange gases. The parenchyma in the diseased portions 24 tends to no longer adequately support the bronchial tree. Without adequate support, the parts of the bronchial tree in the diseased portions 24 collapse which prevents airflow in the diseased portions 24 and reduces the overall airflow capacity in the lungs 10.

During inspiration, the expansion of the lungs 10 can open closed airways in the bronchial tree to allow air to enter the diseased portions 24. However, the air received during inspiration can become trapped within the diseased portions 24 because the air passages are allowed to collapse in the diseased portions 24 during expiration, which can lead to a hyperinflated lung. The trapped air increases the volume of the diseased portion 24, which in turn, prevents the full expansion of functioning portions of the lung 10, thereby reducing the gas exchange occurring within the functioning portions of the lung 10.

Venting the trapped air in the diseased portions 24 during expiration reduces the volume of the diseased portions 24 during inspiration, which in turn, allows the functioning lung portions to expand to a greater extent and increase the gas exchange within the functioning portions of the lung 10.

To vent air that is trapped in the diseased portions 24, openings may be formed in the walls of the air passages of the bronchial tree and stents 28 may be placed within the openings. For example, the stents 28 may be placed in the walls of the lobar bronchi 16 or segmental bronchi 18. The tissue in which the opening is formed may be made of cartilage, smooth muscle, or a combination of both. When placed in the openings, the stents 28 form artificial conduits through the walls of the air passages in the diseased portions 24. The artificial conduits formed by the stents 28 allow the venting or draining of air and other fluids trapped in the diseased lung tissue and surrounding space near the stent 28.

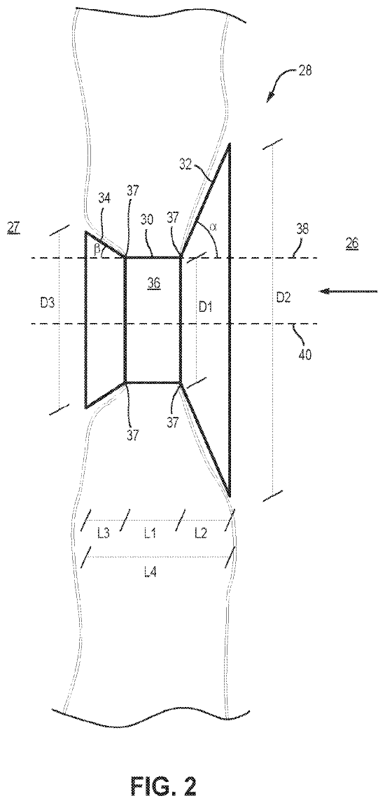

FIG. 2 is a side view of an exemplary stent 28 positioned in a wall of an airway in the lung so that an inlet of the stent 28 extends into the lung parenchyma 26 and the outlet extends into the airway 27. The stent 28 has a hollow structure and includes a middle section 30, an inlet section 32 that is designed to be positioned inside the lung tissue, and an outlet section 34 that is designed to be positioned inside an airway of the lung. The inlet section 32 and the outlet section 34 may be asymmetric. That is, the inlet section 32 and the outlet section 34 may have different sizes and/or different shapes. In addition, the middle section 30, the inlet section 32, and the outlet section 34 together form a central passage 36 through which the trapped air and fluid may be vented and drained.

It is contemplated that the stent 28 may be asymmetrical along its length. For example, the inlet section 32 may be wider than the outlet section 34. The wider inlet section 32 may extend beyond the air passage of the lung and may allow air trapped in lung tissue immediately outside of the air passage to enter the air passage. The wider aspect of the inlet section 32 may also assist in seating the stent 28 in the opening in the air passage. The outlet section 34 may be narrower than the inlet section 32 to reduce the profile of the stent 28 in the air passage which should reduce the amount of mucus collecting on and obstructing the stent 28. Also, the narrower outlet section 34 may reduce the risk that the self-expanding stent will pop off the catheter of the delivery tool with such force that the stent 28 dislodges from the opening in the air passage.

The middle section (or body) 30 of the stent 28 may be in the form of a hollow tube with a circular cross-sectional shape. The interior surface of the middle section 30 may be smooth and may lack any valve devices or protrusions that may disrupt the flow of air through the middle section 30. The length L1 of the middle section 30 may sized to extend across the thickness of the air passage wall and may be within a range of about 3 mm to about 13 mm, about 4 mm to about 6 mm, or about 4 mm to about 5 mm. For example, the length L1 of the middle section 30 may be about 5 mm, about 6.5 mm, or about 8 mm.

It is contemplated that the length L1 of the middle section 30 may be sized to be the exact thickness of the air passage wall (which on average may be 3 mm) so that the stent 28 fits like a rivet. In addition, the length L1 may be sized to account for tissue displaced by the creation of fenestration and may be, for example, within a range of about 6 mm to about 7 mm. It should be understood that the length L1 of the middle section 30 should be long enough so that the inlet and outlet sections 32, 34 can fully deploy and create a sufficient anchoring force to anchor the stent 28 in place.

In addition, the diameter D1 (i.e., the inner diameter of the middle body 30) of the middle section 30 may be consistent along its length and may be within the range i of about 6 mm to about 13 mm, about 8 mm to about 12 mm, or about 5 mm to about 9 mm. For example, the diameter D1 of the middle section 30 may be about 5 mm, about 6.5 mm, or about 8 mm. Alternatively, the diameter D1 of the middle section 30 may increase from one end to the other. For example, the diameter D1 of the middle section 30 may increase from a first end adjacent to the inlet section 32 to a second end adjacent to the outlet section 34 or may increase form the second end to the first end. The range of diameters D1 disclosed above also apply to the configuration with the variable diameter.

In this configuration, the diameter D1 of the middle section 30 of the stent 28 may be selected to be larger relative to its length as compared to a conventional stent. In particular, the ratio of the diameter D1 of the middle section 30 to the length L1 of the middle section 30 may be within the range of 1 to 4.5, 1.5 to 3, or 2 to 2.5 (or the diameter D1 may be 100% to 450% the length L1, 150% to 300% the length L1, or 200% to 250% the length L1). For example, the ratio of the diameter D1 to the length L1 may be 1.6 or 1.3 (or the diameter D1 may be 160% the length L1 or 130% the length L1). The larger diameter reduces the risk of the central passage 36 becoming clogged with mucus, tissue, or other material.

The size of the diameter D1 is large enough to minimize the likelihood of a mucus plug forming in the middle section 30. In addition, the size of the diameter D1 decreases the amount of pressure needed to clear mucus or other bodily fluid out of the stent 28.

It is contemplated that the cross-sectional shape of the middle section 30 may have another shape such as an oval, octagon, square, or other closed shape. It is further contemplated that different portions of the middle section 30 may have different cross-sectional shapes. For example, the portion of the middle section 30 closest to the inlet section 32 may have a circular shape while the portion of the middle section 30 closest to the outlet section 34 may have an oval shape. The transition between shapes may be gradual or abrupt. For stents 28 having a non-circular cross-section the maximum width or diameter D1 of the cross-sectional shape may be within the range of about 6 mm to about 13 mm, about 8 mm to about 12 mm, or about 5 mm to about 9 mm. For example, the maximum width (or maximum diameter) D1 of the middle section 30 may be about 6.5 mm or about 8 mm. In addition, the ratio of maximum width (or maximum diameter) to length may be within the range of 1 to 4.5, 1.5 to 3, or 2 to 2.5.

The middle section 30 may have a bend 37 at each end as the middle section 30 transitions to the inlet section 32 or the outlet section 34. The bend 37 may be abrupt, such as with a radius of curvature of about 1 mm to about 3 mm. Alternatively, the bend 37 may be a gradual transition with a large radius of curvature such as about 10 mm to about 15 mm, about 4 mm to about 9 mm, or about 4 mm to about 15 mm. The bend 37 between the middle section 30 and the outlet section 34 may extend the length L2 of the outlet section 34.

When in the deployed state, the inlet section 32 extends radially outwardly from the middle section 30. Upon being deployed, the inlet section 32 extends from the wall of the air passage into the lung tissue surrounding the wall of the air passage. The fully deployed inlet section 32 may have a frustoconical shape or other shape that is compatible with the cross-sectional shape of the middle section 30.

The inlet section 32 may be in the form of a circumferential flange at an end of the middle section 30 that forms an angle α with a plane 38 that is parallel to the central longitudinal axis 40 of the central passage 36. When fully deployed, the angle α may be between about 85 degrees and about 90 degrees. For example, the angle α may be about 90 degrees when the stent 28 is fully deployed. It is contemplated that the angle α may be constant along the length of the inlet section 32. Alternatively, the angle α may vary along the length of the inlet section 32 (e.g., gradually increase in a direction away from the middle section 30).

The large angle and large diameter allow the inlet section 32 to grip the lung tissue and anchor the stent 28 in place. The larger size and angle allows for greater airflow while also providing a sufficient retaining force that counters the force of a cough or expiration that could dislodge the stent 28. Smaller angles α may increase the likelihood of the stent 28 being dislodged from the lung tissue during use. In addition, diameters that are too large could adversely impact surrounding structures.

In addition, the maximum diameter D2 of the fully deployed inlet section 32 may be in the range of about 10 mm to about 15 mm or about 13 mm to about 14 mm. For example, the diameter D2 may be about 13 mm or about 14 mm. A length L2 of the fully deployed inlet section 32 may be within a range of about 0.5 mm to about 2 mm. For example, the length L2 may be about 1 mm.

The outlet section 34 extends radially outwardly from the middle section 30. Upon being deployed, the outlet section 34 extends into the air passage of the lung. Similar to the inlet section 32, the fully deployed outlet section 34 may have a frustoconical shape or other shape that is compatible with the cross-sectional shape of the middle section 30.

The outlet section 34 may be in the form of a circumferential flange at an end of the middle section 30 that forms an angle β with the plane 38. When fully deployed, the angle β may be between about 45 degrees and about 85 degrees. For example, the angle β may be about 75 degrees when the stent 28 is fully deployed. It is contemplated that the angle β may be constant along the length of the outlet section 34. Alternatively, the angle β may vary along the length of the inlet section 32 (e.g., gradually increase in a direction away from the middle section 30).

The angle R may be equal to or smaller than the angle α. It is contemplated that the ratio between the angle α and the angle β may be within a range of 1 to 2 (or the angle β may be 50% to 100% of the angle α). For example, the ratio between the angle α and the angle β may be 1.2 (or the angle β may be 88% of the angle α).

In addition, the maximum diameter D3 of the fully deployed outlet section 34 may be in the range of about 10 mm to about 15 mm or about 11 mm to about 12 mm. For example, the diameter D3 may be about 11 mm or about 12 mm.

The diameter D3 of the fully deployed Outlet section 34 may be greater than the diameter D2 of the fully deployed inlet section 32. It is contemplated that the ratio between the diameter D2 of the inlet section 32 and the diameter D3 of the outlet section 34 may be between 1 and 1.3 (or the diameter D3 may be 73% to 100% the diameter D2). For example, the ratio between the diameter D2 and the diameter D3 may be 1.2 or 1.3 (or the diameter D3 may be 83% of the diameter D2 or may be 77% the diameter of D3).

A length L3 of the fully deployed outlet section 34 may be within a range of about 0.5 mm to about 2 mm. For example, the length L3 may be about 1.5 mm. The length L3 of the fully deployed outlet section 34 may be greater than the length L2 of the fully deployed inlet section 32. It is contemplated that the ratio of the length L2 to the length L3 may be between 0.25 and 1 (or the length L3 may be 100% to 400% the length L2). For example, the ratio of the length L2 to the length L3 may be 0.67 (or the length L3 may be 150% the length L2).

The relatively short length L3 of the outlet section 34 may reduce the amount of mucus that collects on the stent 28 as air flowing through the airway in the bronchus passes over the outlet section 34. In addition, the relatively shallower angle allows the outlet section 34 to conform to the tapered shape of the airway and minimize granulation. Also, diameters R that are too large could cover or block adjacent airways and block mucus flow.

The relative lengths and relative diameters of the different sections of the stent 28 are selected so that a relatively small portion of the stent 28 protrudes into the airway of the bronchus or bronchi with sufficient force to resist being displaced while at the same time avoiding covering adjacent airways or adversely affecting adjacent tissue. For example, the length L1 of the middle section 30 of the stent 28 may be 150% to 2600% the lengths L2, L3 of each of the inlet and outlet sections 32, 34.

In addition, the diameters D2 and D3 may be sized so that for larger the diameters D1, diameters D2 and D3 are larger so that the ratio of the diameter D1 to the diameter D2 and/or diameter D3 may be the same regardless of the size of the diameter D1. For example, the diameter D1 may be 30% to 85% of the diameters D2 and 23. For example, the diameter D1 may be 35% of the diameter D2 and may be 45% of the diameter D3. The Table 1 below shows the relatives sizes of diameters D1, D2, and D3 as compared to each other and shows the relative size of the diameter D1 compared to the length L1 for two exemplary configurations.

| | | | | Ratio of | Ratio of | Ratio of |
|---|---|---|---|---|---|---|
| Diameter | Length | Diameter | Diameter | Diameter | Diameter | Diameter |
| D1 (mm) | L1 (mm) | D2 (mm) | D3 (mm) | D1 to L1 | D2 to D1 | D3 to D1 |
| 6.5 | 5 | 11 | 12 | 0.77 | 1.69 | 1.85 |
| 8 | 5 | 13 | 14 | 0.63 | 1.63 | 1.75 |

The total length L4 of the stent 28 including the middle section 30, the inlet section 32, and the outlet section 34 may be in a range of about 3.5 mm to about 10 mm. For example, the length L4 may be about 6 mm.

It is contemplated that the surface of the stent may be made of a hydrophobic material or coated with a hydrophobic material to prevent mucus or other bodily fluid from adhering to the stent, which could reduce the size of or clog the bypass airway.

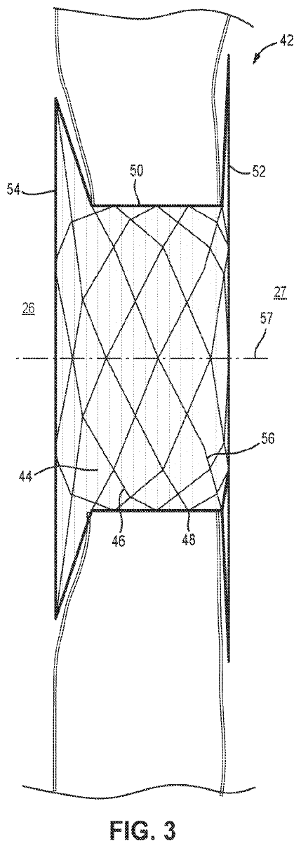

In order to deploy the stent into the lung, the stent may be flexible and compressible so that the stent can be positioned within an insertion tool through small passages and then be secured in the lung. During delivery, the stent is in a compacted configuration, and after deployment, the stent is in an expanded configuration. FIG. 3 shows an exemplary resiliently flexible stent 42 in an expanded configuration. The stent 42 may comprise a membrane 44 and a supporting structure 46. In the extended configuration, the stent 42 has the same shape and dimensions as the stent 28 shown in FIG. 2.

The membrane 44 may form a continuous surface forming the walls of a central passage 48 that vents and drains the trapped air and fluid from the lung tissue into the air passage. The membrane 44 may be non-porous and may be a thin polymer, such as polytetrafluoroethylene (PTFE) or a thin elastomer such as silicone. It is contemplated that the thickness of the membrane 44 may be in a range between about 0.1 mm and 0.5 mm. The material of the membrane 44 may reduce tissue irritation caused by the stent 42 in the opening in the wall of the airway. The membrane 44 may also prevent granulation tissue growth, e.g., scar tissue, from extending through the stent 42 and partially closing the opening. The membrane 44 may also provide a relatively smooth surface for mucus flow. However, some granulation tissue inside airway may be beneficial for keeping the stent 42 attached to the lung tissue and to prevent migration.

It is contemplated that the membrane 44 may optionally only cover the middle section 50. If the inlet section 52 and the outlet section 54 are not covered by the membrane 44, the membrane 44 may be porous or only cover the supporting structure 46. If the inlet section 52 and the outlet section 54 are not in direct contact with lung tissue, the membrane 44 may not be needed to prevent the inlet section 52 and the outlet section 54 from irritating body tissue that would cause growth of scar tissue. It is contemplated that the membrane 44 may be molded or bonded to the supporting structure 46 so that at least a portion of the supporting structure is immersed within or coated on one side by the membrane 44.

The membrane 44 is made of a flexible material that might not be able to maintain is shape of resist the collapse of the central passage 48 without additional support. The supporting structure 46 is provided to support the membrane 44 and form the structural skeleton of the stent 42. It is contemplated that the support structure 46 may comprise a wire mesh 56 or other resiliently compressible structure (i.e., a structure that is compressible but is biased toward the expanded configuration). The wire mesh 56 may be formed from a nitinol shape memory alloy, stainless steel, cobalt-chromium, silicone or other biocompatible material that is resiliently bendable. The wire of the wire mesh 56 may have a thickness that allows for flexibility while also providing sufficient support. For example, the thickness of the wire of the wire mesh 56 may be about 0.2 mm. The supporting structure 46 may be woven from a super elastic shape memory alloy wire and may be arranged to allow the stent 42 to be stretched along a central longitudinal axis 57 to be compressed within a catheter if covered by a sleeve. The supporting structure 46 should have sufficient resiliency to force the stent 42 to expand to its expanded shape when the sleeve is withdrawn from covering the stent. Moreover, the wire mesh 56 should have sufficient resiliency to expand even after being compressed for six months to one year.

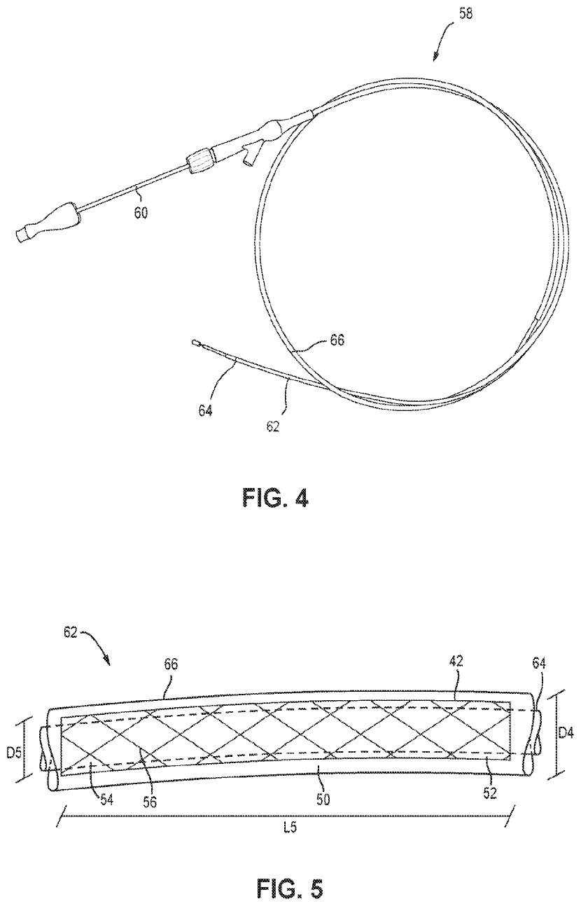

FIG. 4 illustrates an exemplary delivery tool 58 for delivering and installing the stent 42 in a lung. The delivery tool 58 may be, for example, a bronchoscope. The delivery tool 58 may include a proximal end 60 and a distal end 62. A catheter 64 and a sleeve 66 that encompasses the catheter 64 may extend from the proximal end 60 to the distal end 62. The stent 42 may be attached to the delivery tool 58 at the distal end 62. The catheter 64 and the sleeve 66 may be axially movable relative to each other so that the catheter 64 can be exposed by pulling the sleeve 66 toward the proximal end 60 of the delivery tool 58 and/or pushing the catheter 64 away from the proximal end 60. FIG. 4 shows a configuration of the delivery tool 58 in which the catheter 64 is extended beyond the sleeve 66.

FIG. 5 shows the distal end 62 of the delivery tool 58 with the stent 42 positioned on the catheter 64 and encased within the sleeve 66. As can be seen, when the stent 42 is positioned within the delivery tool 58, the catheter 64 extends through the central passage 48. The stent 42 is in a radially compressed configuration to fit within the sleeve 66. That is the diameters of the different sections of the stent 42 are reduced so that the diameters of the different sections of the stent 42 are less than the diameter of sleeve 66. In the radially compressed state, the diameter of the middle section 50, the maximum diameter of the inlet section 52, and the maximum diameter of the outlet section 54 may be the same. Alternatively, the maximum diameters of the inlet and outlet sections 52, 54 may still be greater than the diameter of the middle section 50 when radially compressed and positioned within the sleeve 66. However, all of the diameters of the different sections of the stent 42 are still less than the diameter of the sleeve 66.

The compressed outer diameter D4 of the stent 42 is selected to fit within the sleeve 66. The outer diameter D4 may be in a range of about 1.2 mm to about 2.8 mm, about 1.5 mm to about 2.5 mm, or about 1.3 mm to about 1.7 mm. For example, the outer diameter D4 of the compressed stent 42 may be about 2 mm. The outer diameter D4 of the stent 42 may be selected to allow the stent 42 to slide with respect to the sleeve 66 and particularly to allow the sleeve 66 to be retracted after the stent 42 is positioned in the opening formed in the lung airway. The outer diameter D5 of the sleeve 66 may be in a range of about 2.9 mm to about 2.5 mm. For example, the outer diameter D5 of the sleeve 66 may be about 3 mm. The outer diameter D5 of the sleeve 66 may be selected to fit in a working channel of the delivery tool 58.

In addition, the outer diameter of the catheter 64 may be large enough to frictionally interact with the interior side of the wall of the central passage 36 of the stent 42. The frictional force between the stent 42 and the catheter 64 may be strong enough to resist movement of the stent 42 relative to the catheter 64 while the stent 42 is being deployed. This way, the stent 42 moves with the catheter 64 to the target position during deployment. It should be understood that the frictional force between the stent 42 and the catheter 64 may be low enough so that when the stent 42 is anchored in place, the catheter 64 can be retracted from the stent 42.

The supporting structure 46 may have a lattice-like structure that allows the stent 42 to be radially compressed while at the same time elongating at least the middle section 50 of the stent 42. In such a configuration, at least the membrane 44 is stretched in the axial direction when the stent 42 is radially compressed. In addition, the flanges of the inlet section 52 and the outlet section 54 may be bent or folded to reduce the maximum diameters D2 and D3, which will increase the lengths L2 and L3 of the inlet and outlet sections 52, 54. Accordingly, the length L4 of the stent 42 may be greater when the stent 42 is radially collapsed than when the stent 42 is radially expanded. For example, the length L4 of the stent 42 may increase by 10%, 25%, 30% or 45% or greater when the stent 42 is radially compressed. In addition, because the maximum diameter D2 of the inlets section 52 is greater than the maximum diameter D3 of the outlet section 54, the increase in length L2 of the inlet section 52 when the stent 42 is radially compressed may be greater than the increase in length L3 of the outlet section 54.

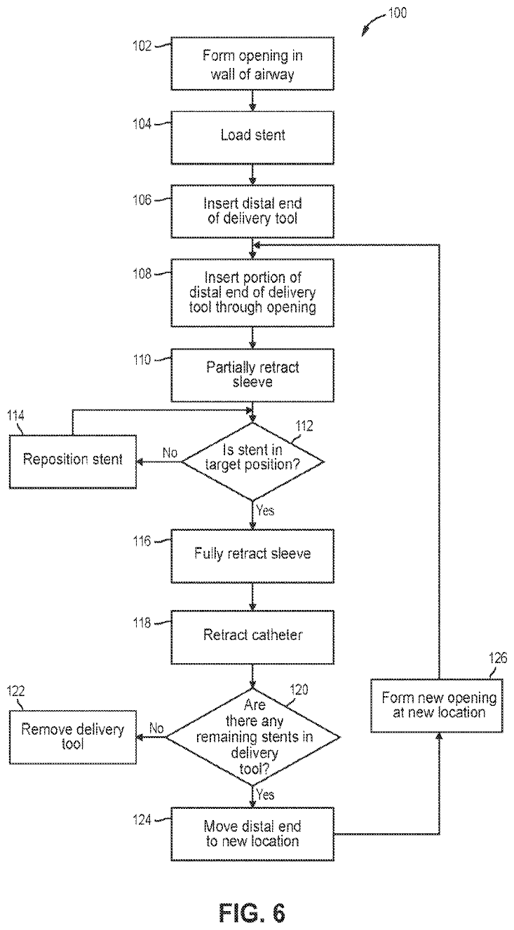

FIG. 6 is a flow chart for an exemplary method 100 for deploying the stent 42 in the lung tissue. The method may begin by forming an opening in a wall of the airway within or adjacent to the diseased lung portion 24 (step 102). The opening may have a cross-section (e.g., diameter) that is smaller than the fully expanded maximum diameter D3 of the outlet section 54 but larger than the diameter D1 of the middle section 50. The opening may be large enough to allow the inlet section 52 to pass through in the compressed state but small enough so that the flange of the inlet section 52 anchors the stent 42 to the wall of the airway and prevents the pressure in the airway from drawing the stent 42 out of the lung tissue.

The stent 42 may be loaded into the distal end of the delivery tool 58 (step 104). As discussed above, the stent 42 may be positioned in the distal end 62 of the delivery tool 58. In addition, it is contemplated that a sequence of stents 42 may be loaded into the distal end 62 of the delivery tool 58 so that multiple stents 42 can be implanted without having to completely remove the delivery tool 58 from the patient's airways.

Once the stent (or stents) 42 is loaded onto the delivery tool 58, the distal end 62 of the delivery tool 58 (with the stent or stents 42) may be inserted into an airway leading to a diseased lung portion 24 (step 106). The distal end 62 may be inserted through the subject's mouth or nasal passage and through the trachea 12, the main bronchus 14, the lobar bronchus 16, the segmental bronchus 18, and ultimately through the bronchioles 20. The distal end 62 may be directed to the opening by an imaging system or other device that allows the user to determine the location of the distal end 62. The delivery tool 58 may also have markings or a signaling system (e.g., tactile, visual, and/or audible) that lets the user know when the distal end 62 has reached its target location (or depth) (e.g., a location adjacent to the opening or partially through the opening). It is contemplated that radiopaque markers may be placed on each end and/or at the center of the stent 42 to allow a user to determine the location of the stent 42 relative to the target location.

Upon reaching the opening, the distal end 62 may be inserted through the opening so that a portion of the inlet section 52 is located within the lung tissue (step 108). When a portion of the inlet section 52 is extended through the opening, the sleeve 66 may be partially retracted to expose the part of the inlet section 52 that is extended through the opening (step 110). It is contemplated that a lubricant may be applied to the inner side of the sleeve 66 and/or an outer surface of the stent 42 to facilitate the sliding movement between the sleeve 66 and the stent 42.

Because only a portion of the inlet section 52 is extended through the opening, the diameter of the opening will prevent the inlet section 52 from being fully extended. At this stage, the flange of the inlet section 52 is only partially extended (or deployed) so that the diameter D2 of the inlet section 52 is between the fully extended maximum diameter and the fully compressed minimum diameter. In addition, when partially deployed, the angle α at which the flange of the inlet section 52 is positioned is less than the maximum angle α of the flange of the inlet section 52 when the inlet section 52 is fully expanded (or deployed). For example, the angle α may be 50% of the maximum angle α when the inlet section 52 is partial deployed of the inlet section 5.

Leaving the flange of the inlet section 52 only partially expanded results in a smaller angle α that does not have the same anchoring force as the fully expanded angle α. Thus, in the partially deployed state, the inlet section 52 allows the stent 42 to move within the opening but provides some resistance to the movement of the stent 42. The result is a more accurate positioning of the stent 42 in the opening.

Once the sleeve 66 has been partially retracted, a determination is made as to whether the stent 42 is in the target position (e.g., the flange of the inlet section 52 is positioned against the outer surface of the wall of the airway and the flange of the outlet section 54 is positioned against the inner surface of the wall of the airway) (step 112). If the stent 42 is not in the target position (step 112: No), the catheter 64 may be moved to reposition the stent 42 (step 114). It should be understood that the sleeve 66 is still covering most of the stent 42 including a portion of the inlet section 52. In addition, the flange of the inlet section 52 may provide an anchoring force that acts against movement of the stent 42 but can be overcome by applying a strong enough pushing or pulling force on the catheter 64. Once the stent 42 has been repositioned, step 112 may be repeated.

If it is determined that the stent 42 is in the target position (step 112: Yes), the sleeve 66 may be fully retracted from the stent 42 (step 116) to allow the stent 42 to expand to a deployed position. For stent 42, the mesh wire 56 is biased to the fully expanded configuration. Thus, the stent 42 automatically expands to the expanded configuration upon the sleeve 66 being retracted from the stent 42.

Some stents might not be biased toward an expanded configuration. For those stents, the catheter 64 may include an expandable balloon positioned between the catheter 64 and the stent. To expand the stent, the balloon may be inflated with air or filled with another fluid. The expansion of the balloon forces the stent to expand and seat in the opening of the airway passage. The expanding balloon is useful for stents that require mechanical force be expanded. After the stent is seated in the opening of the airway passage, the balloon is deflated, released from the stent and the balloon and catheter 64 are retracted from the stent which remains seated in the opening of the airway passage.

After the sleeve 66 is retracted and the stent 42 is anchored in place, the catheter 64 may be retracted from the stent 42 (step 118).

After the catheter 64 has been retracted and the stent 42 is secured in place, it may be determined as to whether there are any other stents 42 remaining in the delivery tool 58 for deployment (step 120). If there are no other stents 42 remaining in the delivery tool 58 (step 120: No), the delivery tool 58 may be removed from the airways (step 122).

If there are more stents 42 remaining in the delivery tool 58, the distal end of the delivery tool 58 may be moved to a new delivery location (step 124), and an opening may be formed in a wall of an air passage at the new location (step 126). Once the new opening is formed, step 108 may be repeated.

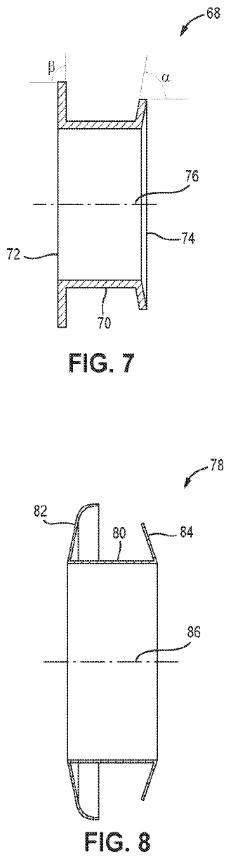

FIG. 7 is a schematic diagram of another exemplary stent 68 configured to be inserted in a hole formed in an air passage of a lung. The stent 68 has a hollow middle section 70, a hollow inlet section 72 and a hollow outlet section 74. The stent 68 may have a support structure and a membrane similar to those discussed above for the stent 42.

The middle section 70 of stent 68 may be cylindrical or have a shape in cross section that is circular, curvilinear, octangular or with straight sides with corners. The length (L) of the middle section 70 is substantially, e.g., 80% to 90%, of the length of the stent 68 along the axis 76. The lengths of the inlet section 72 and the outlet section 74 are nominal, e.g., less than 1 mm, because of the near ninety degree angles α, β, e.g., in a range of 90 degrees to 80 degrees, formed by the inlet and outlet sections 72, 74 with respect to the axis 76. The angle α will always be the same or greater than the angle β.

The cross section of the middle section 70 may have a diameter or largest dimension of about 10 mm or in a range of about 5 mm to about 15 mm, or about 7 mm to about 12 mm. The outer diameter or dimension of the inlet section 72 may be about 16 mm or in a range of about 12 mm to about 20 mm, about 15 mm to about 18 mm or about 16 mm to about 19 mm. The outer diameter or dimension of the middle section 70 is smaller than that of the inlet section 72 by for example 90% to 70%, or 80% to 75%.

FIG. 8 is a schematic diagram of a third exemplary stent 78 configured to be inserted in an opening formed in an air passage of a lung. The stent 78 has a hollow middle section 80, a hollow inlet section 82 and a hollow outlet section 84. The stent 78 may have a support structure and a membrane similar to those discussed above for the stents 42 and 68.

The middle section 80 of stent 78 may be cylindrical or have a shape in cross section that is circular, curvilinear, octangular or with straight sides with corners. The length (L) of the middle section 80 is substantially, e.g., 80% to 90%, of the length of the stent 78 along the axis 86. The lengths of the inlet section 82 and the outlet section 84 are short such as about 2 mm for the inlet section 82 and about 1 mm for the outlet section 84.

The inlet section 82 and outlet section 84 overlap the middle section 80 when in the compressed configuration. The inlet and outlet sections 82, 84 expand when released from the delivery tool 58, such as when the sleeve 66 is retracted. When expanded, the inlet and outlet sections 82, 84 aid in securing the stent 78 to the air passage by pressing against, respectively, the outside surface of the airway and the inside surface. In addition, having the inlet section 82 fold back towards the middle section 80 reduces the distance the inlet section 82 projects into the airway. Reducing the distance the inlet section 82 extends into the airway reduces the tendency of the inlet section 82 to collect mucus and other liquids which can clog the stent.

The cross section of the middle section 80 may have a diameter or largest dimension of about 10 mm or in a range of about 5 mm to about 15 mm, or about 7 mm to about 12 mm. The outer diameter or dimension of the inlet section 82 may be about 16 mm or in a range of about 12 mm to about 20 mm, about 15 mm to about 18 mm or about 16 mm to about 19 mm. The outer diameter or dimension is smaller than that of the inlet section by for example 90% to 70%, or 80% to 75%.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Also, the terms “approximately”, “about”, and “substantially” encompass a range of plus or minus 10%. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

### Elements

10 Lung
12 Trachea
14 Main bronchus
16 Lobar bronchus
18 Segmental bronchus
20 Bronchi
24 Diseased lung portion
26 Lung parenchyma
27 Airway
28 Stent
30 Middle section
32 Inlet section
34 Outlet section
36 Central passage
37 Bend
38 Plane
40 Central longitudinal axis
42 Stent
44 Membrane
46 Supporting Structure
48 Central passage
50 Middle section
52 Inlet section
54 Outlet section
56 Mesh wire
57 Central longitudinal axis
58 Delivery tool
60 Proximal end
62 Distal end
64 Catheter
66 Sleeve
68 Stent
70 Middle section
72 Inlet section
74 Outlet section
76 Axis'
78 Stent
80 Middle section
82 Inlet section
84 Outlet section
86 axis
100 Method
102 Step
104 Step
106 Step
108 Step
110 Step
112 Step
114 Step
116 Step
118 Step
120 Step
122 Step
124 Step
126 Step

1.-23. (canceled)

24. A method for implanting a stent in an airway of a lung, the method comprising:
forming an opening in a wall of an airway of the lung;
advancing a delivery tool through the airway wherein a distal end region of the delivery tool includes a catheter on which the stent, in a compressed configuration, is mounted and a sleeve extending over the catheter and stent;
advancing the distal end region of the delivery tool from the airway and at least partially through the opening in the wall of the airway and into lung tissue adjacent the wall;
positioning the stent in the opening by manipulating the distal end of the delivery tool;
moving the sleeve or advancing the catheter to move a first flange of the stent to extend from the sleeve and be positioned in the lung tissue and to allow the first flange to at least partially expand from the compressed configuration, and
further moving the sleeve or advancing the catheter such that stent is beyond the sleeve and the stent further expands from the compressed configuration into an expanded configuration.

25. The method of claim 24, wherein the step of positioning the stent includes advancing and/or retracting the distal end of the delivery tool to align a middle section of the sent with the opening in the wall.

26. The method of claim 24, wherein the first flange is pulled against the wall of the airway.

27. The method of claim 24, wherein the first flange anchors the stent to the lung tissue or wall to prevent the stent from being sucked into the airway.

28. The method of claim 24, wherein the catheter is retracted from the airway after the stent is in the expanded configuration.

29. The method of claim 24, wherein, while the stent is in the expanded configuration, a maximum diameter of the first flange is greater than a maximum diameter of the second flange.

30. The method of claim 24, wherein the first flange and the second flange are asymmetric.

31. The method of claim 24, wherein the step of positioning includes retracting the catheter with the stent after the first flange is partially expanded to anchor the first flange in the lung tissue.

32. The method of claim 31, wherein the stent is partially movable relative to the opening while the first flange is partially expanded.

33. The method of claim 32, wherein the stent is stationary and fully anchored to the lung tissue when the stent is in the expanded configuration.

34. A method for venting fluid from tissue in a lung, the method comprising:
forming an opening in a wall of a lung airway near the tissue;
inserting a collapsed stent into the opening; and
expanding the stent so that the stent is anchored in the opening and an inlet flange of the stent is in the tissue and an outlet flange of the sent is in the airway,
wherein the opening and the stent provide a pathway for fluid to vent or drain from the lung tissue into the airway.

35. The method of claim 34, wherein the stent is configured to automatically expand when positioned in a target location.

36. The method of claim 34, wherein the inlet flange is anchored to the lung tissue.

37. The method of claim 34, wherein the stent comprises a middle passage between the inlet flange and the outlet flange, and the middle passage is positioned in the opening of the airway.

38. The method of claim 37, wherein the inlet flange has a larger outermost diameter than the outlet flange.

39. The method of claim 34, wherein the stent is biased toward an expanded state.

40. The method of claim 34, wherein at least part of the stent is encased in a wire mesh.
